SEQUENCE LISTING (23 sequences)

SEQ ID NO: 1 - DNA, 255 bp, Bifidobacterium adolescentis
ctccagttggatgcatgtccttctgggaaagattctatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccg

SEQ ID NO: 2 - DNA, 255 bp, Bifidobacterium adolescentis
ctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgc

SEQ ID NO: 3 - DNA, 255 bp, Bifidobacterium bifidum
ttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacct

SEQ ID NO: 4 - DNA, 256 bp, Bifidobacterium dentium
ctccggttggatgcatgtccttccgggaaagattccatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgc

SEQ ID NO: 5 - DNA, 255 bp, Bifidobacterium catenulatum/pseudocatenulatum
ctccgacatgacgcatgtcgtgttgggaaagattcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgc

SEQ ID NO: 6 - DNA, 255 bp, Bifidobacterium catenulatum/pseudocatenulatum
  misc_feature (19)..(19): n is a, c, g, or t
ctccaacatgacgcatgtngtgtcgggaaagattcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgc

SEQ ID NO: 7 - DNA, 255 bp, Bifidobacterium catenulatum/pseudocatenulatum
ctccgactcctcgcatggggtgtcgggaaagattatatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccg

SEQ ID NO: 8 - DNA, 255 bp, Bifidobacterium longum
ctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctc

SEQ ID NO: 9 - DNA, 255 bp, Bifidobacterium adolescentis
ctccgacatgacgcatgtcgtgtcgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgc

SEQ ID NO: 10 - DNA, 255 bp, Bifidobacterium adolescentis
ctccagttgaccgcatggtcctctgggaaagcttttgcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgc

SEQ ID NO: 11 - DNA, 255 bp, Bifidobacterium adolescentis
ctccagttgaccgcatggtcctctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgc

SEQ ID NO: 12 - DNA, 255 bp, Bifidobacterium catenulatum/pseudocatenulatum
tgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaat

SEQ ID NO: 13 - DNA, 18 bp, Bifidobacterium bifidum
tcgcgtcyggtgtgaaag

SEQ ID NO: 14 - DNA, 17 bp, Bifidobacterium bifidum
ccacatccagcrtccac

SEQ ID NO: 15 - DNA, 19 bp, Bifidobacterium longum
cagttgatcgcatggtctt

SEQ ID NO: 16 - DNA, 16 bp, Bifidobacterium longum
tacccgtcgaagccac

SEQ ID NO: 17 - DNA, 21 bp, Bifidobacterium bifidum
ccacatgatcgcatgtgattg

SEQ ID NO: 18 - DNA, 19 bp, Bifidobacterium bifidum
ccgaaggcttgctcccaaa

SEQ ID NO: 19 - DNA, 17 bp, Bifidobacterium catenulatum/pseudocatenulatum
cggatgctccgactcct

SEQ ID NO: 20 - DNA, 18 bp, Bifidobacterium catenulatum/pseudocatenulatum
cgaaggcttgctcccgat

SEQ ID NO: 21 - DNA, 19 bp, Bifidobacterium adolescentis
ctccagttggatgcatgtc

SEQ ID NO: 22 - DNA, 18 bp, Bifidobacterium adolescentis
tccagttgaccgcatggt

SEQ ID NO: 23 - DNA, 18 bp, Bifidobacterium adolescentis
cgaaggcttgctcccagt

(19) United States
(12) Patent Application Publication
(10) Pub. No.: US 2012/0301435 A1
(43) Pub. Date: Nov. 29, 2012

(54) PROBIOTIC BIFIDOBACTERIAL COMPOSITION IN ACCORDANCE WITH SECRETOR BLOOD GROUP STATUS

(75) Inventors: Pirjo Wacklin, Helsinki (FI); Jaana Matto, Helsinki (FI); Harri Makivuokko, Helsinki (FI)

(73) Assignee: SUOMEN PUNAINEN RISTI VERIPALVELU, Helsinki (FI)

(21) Appl. No.: 13/519,493

(22) PCT Filed: Dec. 28, 2010
(86) PCT No.: PCT/FI10/51094

§ 371 Date: Jun. 27, 2012

Related U.S. Application Data
(63) Continuation-in-part of application No. 12/843,405, filed on Jul. 26, 2010.

(30) Foreign Application Priority Data
Dec. 28, 2009 (FI) 20096402

(51) Int. Cl. A61K 35/74; A61P 31/04; C12Q 1/04; A61P 37/08; A61P 1/12; C12N 1/20; A61P 29/00; A61P 11/00

(52) U.S. Cl. 424/93.4; 435/252.1; 435/34

(57) ABSTRACT

The present invention relates to a microbial or probiotic composition which is tailored based on the spectrum of bifidobacteria found in the intestine of at least one individual with secretor blood group phenotype but not commonly found in individuals of non-secretor blood group phenotype. The present invention further relates to a method of tailoring a microbial or probiotic composition based on the bifidobacteria found in the intestine of at least one individual with secretor blood group phenotype but not commonly found in individuals non-secretor blood group phenotype.

### FIELD OF THE INVENTION

The present invention relates to a microbial or probiotic composition which is tailored based on the spectrum of bifidobacteria found in the intestine of at least one individual with secretor blood group phenotype but not commonly found in individuals of non-secretor blood group phenotype. The present invention further relates to a method of tailoring a microbial or probiotic composition based on the bifidobacteria found in the intestine of at least one individual with secretor blood group phenotype but not commonly found in individuals non-secretor blood group phenotype. Also, the invention relates to the use of prebiotics, molecular compounds or additional supportive bacteria strains, to augment the growth or functionality of bifidobacteria in the intestine. The present invention also relates to use of the secretor status of an individual as a criterion for bifidobacteria-enriched probiotic supplementation. The present invention relates also to method of assessing the need of an individual for bifidobacteria-enriched probiotic supplementation by determining the secretor status of the individual. Additionally, the invention relates to a probiotic composition for use in treating and/or preventing diseases such as inflammatory bowel disease, diarrhoea, respiratory tract infections, irritable bowel syndrome and/or atopy or allergy.

### BACKGROUND OF THE INVENTION

Bifidobacteria comprise the predominant intestinal microbiota in infants and they are adundant also in the adult population comprising up to 10% of the normal intestinal microbiota, although their numbers start to decline in the elderly. An individual is typically colonised with 1-4 bifidobacterial species (Mättö et al. J Appl Microbiol 2004; 98:459-470). In addition to the individual variation, composition of bifidobacterial species also varies between different age groups. B. longum biovar infantis, B. breve and B. bifidum are the most prevalent species in infants and B. longum biovar longum, B. adolescentis, B. bifidum and B. catenulatum in adults. Variation in the number of bifidobacteria (Mueller at al. Appl Environ Microbiol 2006; 72:1027-1033) and composition of species (Mättö et al. 2004) between geographic regions has also been reported. Bifidobacteria are generally considered as health promoting bacteria and an increase in bifidobacterial numbers in the intestine has been used as an end-point in intervention studies with intestinal health-targeted products such as probiotics and prebiotics.

Bifidobacterium spp. strains are used as probiotics. However, due to technological challenges related to stability of the genus, fairly few distinct species and strains, mainly B. animalis subps. lactis, are available on the current market. Bifidobacteria or bifidobacteria-containing strain mixtures have shown promising results e.g. in alleviation of the symptoms of irritable bowel syndrome (Brenner & Chey, Rev Gastroenterol Disord. 2009; 9:7-15), inflammatory bowel disease (Macfarlane et al. Crit Rev Clin Lab Sci 2009; 46: 25-54), diarrhoea (Chouraqui et al. J Pediatr Gastroenterol Nutr 2004; 38: 242-3), atopic eczema (Yoo et al. Proc Am Thorac Soc 2007; 4: 277-282) and common cold (de Vrese et al. Clin Nutr 2005; 24: 479-80). Another challenge in addition to the above-mentioned stability problems, is the fact that a proportion of the study subjects usually have not responded to test probiotics or prebiotics (Fuccio et al. J Clin Gastroenterol 2009; 43: 506-513; Fujimori et al. J Gastroenterol Hepatol 2007; 22: 1199-1204). These individuals are often said to be ‘non-responders’. No reason behind the non-responsiviness is known.

The primary site of colonization of bifidobacteria is the colon, but they are also present in the oral cavity and have been isolated from human milk (Martin et al. Appl Environ Microbiol. 2009, 75:965-9). The major energy sources of bifidobacteria are non-digestible dietary carbohydrates and endogenous mucus. They are capable of degrading various oligosaccharides including human milk oligosaccharides and complex carbohydrates present in mucus as substrates. Several bifidobacteria have been shown to adhere to intestinal mucus (He et al. Microbiol Immunol 2001, 45, 259-262). Adhesion of Bifidobacterium bifidum to mucus has been shown to increase by supplementation of fucose (Guglielmetti et al. Curr Microbiol. 2009; 59:167-72). The vast variety and spectrum of microbial strains and species in the gut of mammals, including man and the findings demonstrating that the composition of microbial species in the gut will not directly predict their functional outcome have indicated that predicting the functionality of single probiotic or normal flora species is difficult (Tap et al. Environm Microbiol 2009, 11, 2574-2584). The complexity of the ecosystem is simply too vast. The role of host genetic factors in determining the composition of normal gut microbiota is also poorly understood.

Binding to blood group antigens has been reported for certain single pathogenic species of bacteria and viruses. In particular, Helicobacter pylori binds to the Lewis b (Le^{b}) antigen in stomach (Boren et al. Science 1993; 262: 1892-1895) and Norovirus binds to ABH ja Le^{b} antigens (Huang et al. J. Virol. 2005; 79: 6714-22). Streptococcus pneumoniae has ability to bind A and B blood group antigens and utilise the glycans (Higgins et al. J Mol Biol. 2009; 388: 299-309).

The blood group antigens are not present in the mucus of all individuals. These individuals, said to have ‘non-secretor’ blood group, do not have the functional FUT2 gene needed in the synthesis of secreted blood group antigens (Henry et al. Vox Sang 1995; 69(3):166-82), and thus they do not secrete ABH antigens in secretions and on mucosa. Those with blood group ‘secretor’ have the antigens on mucosa. In most populations, the frequency of non-secretor individuals is substantially lower than that of secretor status, about 15-26% of Scandinavians are classified as non-secretors (Eriksson et al. Ann Hum Biol. 1986; 13(3):273-85). The secretor/non-secretor status can be regarded as a normal blood group system and the phenotype can be determined using standard blood banking protocols (Henry et al. 1995). The genotype, that is, the major mutation in the FUT2 gene causing the non-secretor (NSS) phenotype in the European populations (Silva et al. Glycoconj 2010; 27:61-8) has been identified. Non-secretor phenotype has been demonstrated to be genetically associated for example, with an increased risk for Crohn's disease (McGovern et al. Hum Molec Genet 2010; 19: 3468-76.), with high vitamin B12 levels in the blood (Tanaka et al Am J Hum Genet 2009; 84: 477-482), with susceptibility to HI virus infection (Ali et al J Infect Dis 2000; 181: 737-739), with experimental vaginal candidiasis (Hurd and Domino Infection Immunit 2004; 72: 4279-4281), with an increased risk for asthma (Ronchetti et al. Eur Respir J 2001; 17: 1236-1238), with an increased risk for urinary tract infections (Sheinfeld et al N Engl J Med 1989; 320: 773-777), with an increased risk for ETEC caused diarrhea (Ahmed et al. Infect Immun. 2009; 77: 2059-64) and with an animal hemorrhagic disease virus (Guillon et al. Glycobiology 2009; 19: 21-28).

### BRIEF DESCRIPTION OF THE INVENTION

An object of the present invention relates to a microbial or probiotic composition which is tailored based on the spectrum of bifidobacteria found in the intestine of at least one individual with secretor blood group phenotype but not commonly found in individuals of non-secretor blood group phenotype. Another object of the present invention is a method of tailoring a microbial or probiotic composition based on the bifidobacteria found in the intestine of at least one individual with secretor blood group phenotype but not commonly found in individuals non-secretor blood group phenotype. Also, an object of the invention is the use of prebiotics, molecular compounds or additional supportive bacteria strains, to increase the number of, and/or to augment the growth and/or functionality of the said microbial or probiotic composition in the intestine.

A further object of the invention is use of secretor blood group status of an individual as a criterion for bifidobacteria-enriched probiotic supplementation. An additional object of the present invention relates to a method of assessing the need of an individual for bifidobacteria enriched probiotic supplementation by determining the secretor status of the individual.

A further object of the present invention is a use of the secretor blood group status of an individual in estimating a dose of bifidobacteria supplementation needed for a desired effect. Another further object of the present invention is to provide a method of identifying an individual at risk for suffering from a gastrointestinal disorder by determining the secretor status of said individual.

Additional objects of the invention are probiotic compositions for use in treating and/or preventing diseases such as inflammatory bowel disease, diarrhoea, respiratory tract infections, irritable bowel syndrome and/or atopy/allergy. These diseases or disorder are considered to be related to unbalanced mucosal microbiota in an individual.

The invention is based on the observation that the individuals with non-secretor blood group phenotype have a reduced amount and a reduced diversity of bifidobacteria in their intestinal bacterial population as compared to those with the secretor phenotype. In addition, the invention is based on the observation that the bifidobacterial population of the non-secretor individuals show an altered functionality e.g. reduced survival in the harsh conditions in the upper gastrointestinal tract conditions. This observation can be used as a basis for targeted modulation of the bifidobacterial intestinal population in an individual, especially in a non-secretor individual, in order to result in the higher diversity and/or amount of bifidobacteria species or strains. In other words, the modulation aims to diversity and/or amount of bifidobacterium in non-secretors that were alike those found typically in secretor individuals. Accordingly, the current invention provides a novel and effective means for optimizing the bacterial, especially bifidobacterial content of a probiotic composition. Such a composition is especially effective for use in individuals with the non-secretor blood group phenotype.

The objects of the invention are achieved by the methods and the uses set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 shows a DGGE gel image of bifidobacterial diversity on faecal samples of 7 non-secretor and 7 secretor individuals. M=marker. Each lane represents a single sample.

FIG. 2 illustrates the three-dimensional PCA plot based on the DGGE analysis of the bifidobacterial profiles.

FIG. 3 illustrates PCA biplot of bifidobacterial DGGE profiles showing the DGGE band positions, which most significantly contributed to the first and the second principal components explaining together 56.3% of the variance. Insert figure indicates the band positions, which contributed the principal component most. Non-secretor samples are indicated with dot, non-secretor with star and samples of unknown secretor status with square.

FIG. 4 illustrates the Shannon diversity Index based on bifidobacterial DGGE profiles test between secretor and non-secretor individuals. P-value for t-test between non-secretor and secretor individuals is shown.

FIG. 5 illustrates the identity of the band positions of Bifidobacteria-DGGE gels based on Blast search of the sequences. The excised and sequenced bands are marked with numbers. The bold letters show band positions, which were either absent or detected rarely in non-secretors. The identity of band positions is shown in the side of the gels with arrows and the colours of the numbers indicate the bands belonging to the same band position and having identical sequences: band position 26.6% (B. adolescentis) contains sequenced bands 15, 24, 27 and 29; band position 29.7% (B. bifidum) contains sequenced bands 6, 16, 20 and 32; band position 53.5% (B. lognum) contains sequenced bands 1, 3, 7, 9, 12, 21 and 33; band position 55.0% (Bifidobacterium sp.) contains sequenced bands 4 and 18; band position 62.2% (uncultured bifidobacterium) contains sequenced bands 1, 5, 13, 19, 25, 31 and 37; band position 63.7% (B. catenulatum/pseudocatenulatum) contains sequenced bands 22 and 34. Identity of the band positions, which were based on single sequence were following (in black): 8=Bifidobacterium sp. (B. catenulatum), 11=B. adolescentis, 17=uncultured bifidobacterium (B. ruminantium), 30=uncultured bifidobacterium (B. adolescentis), 36=uncultured bifidobacterium (B. ruminantium). The strain name in the parentheses indicates the closest cultivated relative of the sequence, if available.

FIG. 6 shows an image of the normalised DGGE profiles for non-secretor individuals, secretor individuals and individuals with unknown secretor status. Numbers in grey boxes and vertical lines indicate the band positions and star symbol on vertical line indicates that band was binned to the band positions.

FIG. 7 illustrates the PCA plot of bifidobacterial DGGE profiles showing the clustering of the samples from non-secretor (n=14) and secretor (n=57) individuals (left) and the DGGE bands, which most significantly contributed to the first and the second principal components (right). The samples from non-secretor individuals formed separate cluster (indicated by circle) within secretor samples.

FIG. 8 illustrates the bifidobacterial diversity (A) and richness (B) in non-secretor and secretor individuals. Significant differences between non-secretor and secretor individuals as tested using the ANOVA are indicated. The samples with no bifidobacterial amplification were excluded from the analysis (one non-secretor individual and six secretor individuals).

FIG. 9 illustrates the detection frequencies (left) and Box and whisker plots (right) of bacteria, bifidobacteria, and bifidobacterial groups in non-secretor (14) and secretor (57) individuals quantified by qPCR. The significant differences between non-secretor and secretors in the Wilcoxon test are indicated.

### DETAILED DESCRIPTION OF THE INVENTION

As bifidobacteria comprise the predominant intestinal microbiota in infants and are adundant also in the adult population, they are considered as essential for maintaining and/or promoting health of an individual. High bifidobacterium diversity in the gut is beneficial for the health of an individual, because bifidobacteria can, for example, prevent adhesion of adverse microbes on gut epithelium and prevent their colonisation in the intestine. They may also modulate the immune response of the host.

The present invention is based on the finding that the non-secretor individuals have a reduced diversity of bifidobacteria and lack several species/genotypes of Bifidobacterium in their intestinal bacterial population. Furthermore, the present invention is based on the finding that the bifidobacterial population of non-secretor individuals has an altered functionality e.g. survival in the upper gastrointestinal tract conditions. These findings can be used as a basis for targeted modulation of the bifidobacterial population in the non-secretor individuals by using probiotic compositions enriched with species/genotypes found in secretor individuals and as a criterion for bifidobacteria enriched probiotic supplementation. Further, these findings can be used in designing probiotic compositions targeted for the treatment and/or prevention of diseases such as inflammatory bowel disease, diarrhoea, respiratory tract infections, irritable bowel syndrome and/or atopy/allergy or the symptoms thereof.

Denaturating Gradient Gel Electrophoresis, DGGE, is a method of choice to detect differences in spectrum or abundance of different bacterial genotypes. In the method, specific PCR primers are designed so that in each experimental setting, only the desired bacterial group or groups are analysed. The differences in band positions and/or their occurrence and/or intensity indicate differences in bacterial compositions between faecal samples. Base composition of the PCR amplified fragment determinates the melting and, thus the mobility of the fragment in the denaturing gradient in gel. The final position of the fragment in gel is consequently specified by the DNA sequence of the fragment, the applied denaturing gradient and the electrophoresis running conditions. The optimised running conditions and denaturing gradient of the gels for the bacterial groups used in this invention are described in Examples. The position of each fragment, the “band position”, between different gel runs are normalised by using standards. The band position is indicated relative to length of the gel, the top being 0% and the bottom edge being 100%.

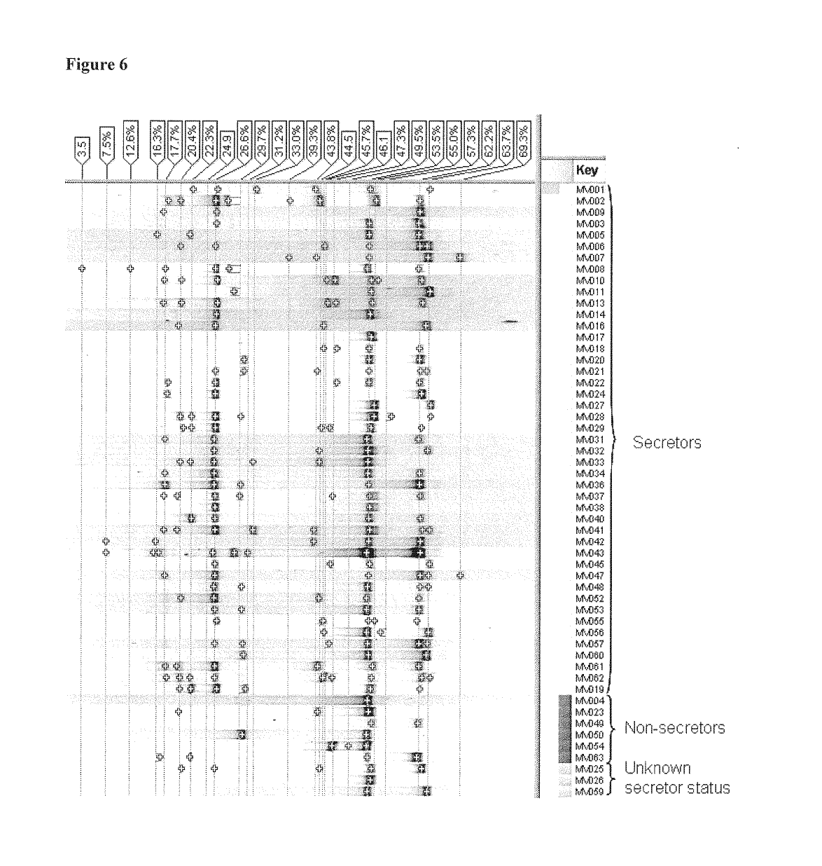

In connection with the present invention, the terms DGGE genotype and bacterial genotype refer to those strains having the same “band position” in the relevant DGGE analysis. Each genotype or a group of closely-related genotypes can be presented as a “band position”. In the present invention, each band position refers to the band positions of the given %-value +/−1% unit, i.e. 25.30% refers to any value between 24.30% and 26.30%, when analysed using the methodology described above. It is noted than depending on the exact conditions the nominant %-value can vary; the relative position of the band to the relevant standard is important. Bifidobacterium DGGE genotypes that were found to be present in secretor individual and absent or at least not commonly found in non-secretor individuals are listed in Table 1. The band positions are presented in detail in FIG. 6.

TABLE 1
Bifidobacterium genotypes that were found to be
present in secretor individual and absent or at least
not commonly found in non-secretor individuals

| Band position | Genotype name |
|---|---|
| 7.5% | Bifidobacterium genotype 1 |
| 3.5% | Bifidobacterium genotype 2 |
| 12.6% | Bifidobacterium genotype 3 |
| 17.7% | Bifidobacterium genotype 5 |
| 24.9% | Bifidobacterium genotype 8 |
| 26.6% | Bifidobacterium adolescentis |
| 31.2% | Bifidobacterium genotype 9 |
| 33.0% | Bifidobacterium genotype 10 |
| 39.3% | Bifidobacterium genotype 11 |
| 44.5% | Bifidobacterium genotype 13 |
| 45.7% | Bifidobacterium genotype 14 |
| 46.1% | Bifidobacterium genotype 15 |
| 57.3% | Bifidobacterium genotype 19 |
| 63.7% | B. catenulatum/pseudocatenulatum |
| 69.3% | Bifidobacterium genotype 21 |

Bifidobacterium DGGE genotypes that were found to be present at least in one non-secretor individual are listed below in Table 2. The band positions are presented in detail in FIG. 6.

TABLE 2
Bifidobacterium genotypes that were found to be
present at least in one non-secretor individual

| Band position | Genotype name |
|---|---|
| 16.3% | Bifidobacterium genotype 4 |
| 20.4% | Bifidobacterium genotype 6 |
| 22.3% | Bifidobacterium genotype 7 |
| 29.7% | Bifidobacterium bifidum |
| 43.8% | Bifidobacterium genotype 12 |
| 47.3% | Bifidobacterium genotype 16 |
| 49.5% | Bifidobacterium genotype 17 |
| 55.0% | Bifidobacterium genotype 18 |
| 62.2% | Bifidobacterium genotype 20 |
| 53.5% | B. lognum |

Based on the DGGE genotypes and isolated strains the inventors were able to identify the bifidobacteria compositions in non-secretor and secretor individuals in more details, that is, to identify the species and diagnostic 16S rRNA nucleotide sequence for band positions. Briefly, the inventors excised the band positions from DGGE gels showing the profiles of faecal samples, sequenced the DNA fragments in the bands and searched for their closest relatives in the sequence databases. In addition, strains isolated from the faecal samples of the non-secretor and secretor individuals were analysed in DGGE in order to screen strains with similar 16S rRNA gene fragment melting behaviour (i.e. sequence) than the observed DGGE bands of faecal samples. Based on the sequences in the DGGE bands and bifidobacterial stains with corresponding band, the bands and further band positions were related to bifidobacterial species. The bifidobacterial species and the diagnostic 16S rRNA fragment sequences of the genotypes detected in non-secretor individuals and only secretor individuals are showed in Tables 3 and 4, respectively.

Bifidobacterium strains that were found to be present at least in one non-secretor individual are listed below in Table 3.

TABLE 3
Bifidobacterium strains that were found to be present at least in
one non-secretor individual

| Identification of the | |
|---|---|
| strains | Sequence 5′->3′ |
| B. adolescentis | No sequence. Strains having band in this |
| (genotype 4) | position (16.3%) have always another band |
| | in position 62.2%. |
| B. adolescentis | No sequence. Strains having band in this |
| (genotype 6) | position (20.4%) have always another band |
| | in position 26.6% |
| B. adolescentis | CTCCAGTTGGATGCATGTCCTTCTGGGAAAGATTCTATCGGTATGG- |
| (genotype 7) | GATGGGGTCGCGTCCTATCAGCTTGATGGCGGGGTAACGGCCCAC- |
| | CATGGCTTCGACGGGTAGCCGGCCTGAGAGGGCGACCGGCCACATT- |
| | GGGACTGAGATACGGCCCAGACTCCTACGGGAGGCAGCAGTGGGGAA- |
| | TATTGCACAATGGGCGCAAGCCTGATGCAGCGACGCCGCGTGCGG- |
| | GATGACGGCCTTCGGGTTGTAAACCG |
| | (SEQ ID NO: 1) |
| B. catenulatum/ | No sequence. Strains having band in this |
| pseudocatenulatum | position (24.9%) have always another band |
| (genotype 8) | in position 44.5%. |
| B. adolescentis | CTCCAGTTGGATGCATGTCCTTCTGGGAAAGATTCATCGGTATGGGATGG |
| | GGTCGCGTCCTATCAGCTTGATGGCGGGGTAACGGCCCACCATGGCTTC |
| | GACGGGTAGCCGGCCTGAGAGGGCGACCGGCCACATTGGGACTGAGAT |
| | ACGGCCCAGACTCCTACGGGAGGCAGCAGTGGGGAATATTGCACAATGG |
| | GCGCAAGCCTGATGCAGCGACGCCGCGTGCGGGATGACGGCCTTCGGG |
| | TTGTAAACCGC |
| | (SEQ ID NO: 2) |
| B. bifidum | TTCCACATGATCGCATGTGATTGTGGGAAAGATTCTATCGGCGTGGGATG |
| | GGGTCGCGTCCTATCAGCTTGTTGGTGAGGTAACGGCTCACCAAGGCTT |
| | CGACGGGTAGCCGGCCTGAGAGGGCGACCGGCCACATTGGGACTGAGA |
| | TACGGCCCAGACTCCTACGGGAGGCAGCAGTGGGGAATATTGCACAATG |
| | GGCGCAAGCCTGATGCAGCGACGCCGCGTGAGGGATGGAGGCCTTCGG |
| | GTTGTAAACCT |
| | (SEQ ID NO: 3) |
| B. dentium | CTCCGGTTGGATGCATGTCCTTCCGGGAAAGATTCCATCGGTATGGGAT |
| (genotype 11) | GGGGTCGCGTCCTATCAGCTTGATGGCGGGGTAACGGCCCACCATGGCT |
| | TCGACGGGTAGCCGGCCTGAGAGGGCGACCGGCCACATTGGGACTGAG |
| | ATACGGCCCAGACTCCTACGGGAGGCAGCAGTGGGGAATATTGCACAAT |
| | GGGCGCAAGCCTGATGCAGCGACGCCGCGTGCGGGATGGAGGCCTTCG |
| | GGTTGTAAACCGC |
| | (SEQ ID NO: 4) |
| B. catenulatum/ | CTCCGACATGACGCATGTCGTGTTGGGAAAGATTCATCGGTATGGGATG |
| pseudocatenulatum | GGGTCGCGTCCTATCAGGTAGTCGGCGGGGTAACGGCCCACCGAGCCT |
| (genotype 12) | ACGACGGGTAGCCGGCCTGAGAGGGCGACCGGCCACATTGGGACTGAG |
| | ATACGGCCCAGACTCCTACGGGAGGCAGCAGTGGGGAATATTGCACAAT |
| | GGGCGCAAGCCTGATGCAGCGACGCCGCGTGCGGGATGACGGCCTTCG |
| | GGTTGTAAACCGC |
| | (SEQ ID NO: 5) |
| B. catenulatum/ | CTCCAACATGACGCATGTNGTGTCGGGAAAGATTCATCGGTATGGGATG |
| pseudocatenulatum | GGGTCGCGTCCTATCAGGTAGTCGGCGGGGTAACGGCCCACCGAGCCT |
| (genotype 13) | ACGACGGGTAGCCGGCCTGAGAGGGCGACCGGCCACATTGGGACTGAG |
| | ATACGGCCCAGACTCCTACGGGAGGCAGCAGTGGGGAATATTGCACAAT |
| | GGGCGCAAGCCTGATGCAGCGACGCCGCGTGCGGGATGACGGCCTTCG |
| | GGTTGTAAACCGC |
| | (SEQ ID NO: 6) |
| B. catenulatum/ | CTCCGACTCCTCGCATGGGGTGTCGGGAAAGATTATATCGGTATGGGAT |
| pseudocatenulatum | GGGGTCGCGTCCTATCAGGTAGTCGGCGGGGTAACGGCCCACCGAGCC |
| (genotype 16) | TACGACGGGTAGCCGGCCTGAGAGGGCGACCGGCCACATTGGGACTGA |
| | GATACGGCCCAGACTCCTACGGGAGGCAGCAGTGGGGAATATTGCACAA |
| | TGGGCGCAAGCCTGATGCAGCGACGCCGCGTGCGGGATGACGGCCTTC |
| | GGGTTGTAAACCG |
| | (SEQ ID NO: 7) |
| Genotype 17 | |
| B. longum | CTCCAGTTGATCGCATGGTCTTCTGGGAAAGCTTTCGCGGTATGGGATG |
| | GGGTCGCGTCCTATCAGCTTGACGGCGGGGTAACGGCCCACCGTGGCTT |
| | CGACGGGTAGCCGGCCTGAGAGGGCGACCGGCCACATTGGGACTGAGA |
| | TACGGCCCAGACTCCTACGGGAGGCAGCAGTGGGGAATATTGCACAATG |
| | GGCGCAAGCCTGATGCAGCGACGCCGCGTGAGGGATGGAGGCCTTCGG |
| | GTTGTAAACCTC |
| | (SEQ ID NO: 8) |
| B. adolescentis | CTCCGACATGACGCATGTCGTGTCGGGAAAGATTCATCGGTATGGGATG |
| (genotype 18) | GGGTCGCGTCCTATCAGCTTGATGGCGGGGTAACGGCCCACCATGGCTT |
| | CGACGGGTAGCCGGCCTGAGAGGGCGACCGGCCACATTGGGACTGAGA |
| | TACGGCCCAGACTCCTACGGGAGGCAGCAGTGGGGAATATTGCACAATG |
| | GGCGCAAGCCTGATGCAGCGACGCCGCGTGCGGGATGACGGCCTTCGG |
| | GTTGTAAACCGC |
| | (SEQ ID NO: 9) |
| B. adolescentis | CTCCAGTTGACCGCATGGTCCTCTGGGAAAGCTTTTGCGGTATGGGATG |
| (genotype 20) | GGGTCGCGTCCTATCAGCTTGATGGCGGGGTAACGGCCCACCATGGCTT |
| | CGACGGGTAGCCGGCCTGAGAGGGCGACCGGCCACATTGGGACTGAGA |
| | TACGGCCCAGACTCCTACGGGAGGCAGCAGTGGGGAATATTGCACAATG |
| | GGCGCAAGCCTGATGCAGCGACGCCGCGTGCGGGATGACGGCCTTCGG |
| | GTTGTAAACCGC |
| | (SEQ ID NO: 10) |
| Genotype 21 | |

Bifidobacterium strains that were found to be present in secretor individuals and absent in non-secretor individuals are listed in Table 4.

TABLE 4
Bifidobacterium strains that were found to be present in secretor
individuals and absent in non-secretor individuals

| Identification of | |
|---|---|
| the strains | Sequence 5′->3′ |
| Genotype 2 | |
| Genotype 1 | |
| Genotype 3 | |
| B. adolescentis | no sequence. Strains having band in this position |
| (genotype 5) | (17.70%) have always another band in position |
| | 26.6% and in 62.2% |
| Genotype 10 | |
| Genotype 22 | |
| B. adolescentis | CTCCAGTTGACCGCATGGTCCTCTGGGAAAGATTCATCGGTATGGGAT |
| (genotype 14) | GGGGTCGCGTCCTATCAGCTTGATGGCGGGGTAACGGCCCACCATGG |
| | CTTCGACGGGTAGCCGGCCTGAGAGGGCGACCGGCCACATTGGGAC |
| | TGAGATACGGCCCAGACTCCTACGGGAGGCAGCAGTGGGGAATATTG |
| | CACAATGGGCGCAAGCCTGATGCAGCGACGCCGCGTGCGGGATGAC |
| | GGCCTTCGGGTTGTAAACCGC |
| | (SEQ ID NO: 11) |
| Genotype 15 | |
| Genotype 19 | |
| B. catenulatum/ | TGCGGGATGACGGCCTTCGGGTTGTAAACCGCTTTTGATCGGGAGCA |
| pseudocatenulatum | AGCCTTCGGGTGAGTGTACCTTTCGAATAAGCACCGGCTAACTACGTG |
| | CCAGCAGCCGCGGTAATACGTAGGGTGCAAGCGTTATCCGGAATTATT |
| | GGGCGTAAAGGGCTCGTAGGCGGTTCGTCGCGTCCGGTGTGAAAGTC |
| | CATCGCTTAACGGTGGATCTGCGCCGGGTACGGGCGGGCTGGAGTG |
| | CGGTAGGGGAGACTGGAAT |
| | (SEQ ID NO: 12) |
| Genotype 23 | |

The term ‘probiotic’ here refers to any bacterial species, strain or their combinations, with health supportive effects, not limited to currently accepted strains or to intestinal effects. The term ‘prebiotic’ here refers to any compound, nutrient, or additional microbe applied as a single additive or as a mixture, together with probiotics or without probiotics, in order to augment a desired probiotic health effect or to stimulate the growth and activity of those bacteria in the digestive system which are assumed to be beneficial to the health of the body.

The present invention relates to a microbial or probiotic composition which is tailored based on the spectrum of bifidobacteria found in the intestine of at least one individual with secretor blood group phenotype but not commonly found in the intestine of an individual with non-secretor blood group phenotype. In one embodiment, the probiotic composition comprises at least one of the strains listed in Table 4. In another embodiment, the probiotic composition comprises two or more of the strains listed in Table 4. Accordingly, optional embodiments of the invention are probiotic compositions comprising for example three or four of the strains listed in Table 4. In yet another embodiment, the probiotic composition comprises Bifidobacterium bifidum together with one or more of the strains listed in Table 4. Accordingly, optional embodiments of the invention are probiotic compositions comprising Bifidobacterium bifidum and for example two or three of the strains listed in Table 4.

In a further embodiment, the microbial or probiotic composition of the invention comprises at least one of the strains listed in Table 1. In an even further embodiment, the probiotic composition comprises two or more of the strains listed in Table 1. Accordingly, optional embodiments of the invention are probiotic compositions comprising for example three, four or five of the strains listed in Table 1. In yet further embodiment, the probiotic composition comprises Bifidobacterium bifidum and one or more of the strains listed in Table 1. Accordingly, optional embodiments of the invention are probiotic compositions comprising Bifidobacterium bifidum and for example two, three or four of the strains listed in Table 1.

In the present invention the phrase “bifidobacterium genotypes that are not commonly found in non-secretor individuals” refers to bifidobacteria species or strains that are not typical to colonize the intestine of a non-secretor individual and/or not typical to be found from the intestine of a non-secretor individual. In the present invention the term “not commonly found” refers to frequency of typically less than 10%, such as of 5-10%, among non-secretors to have detectable levels of said bifidobacterium species or strain.

The present invention further relates to a method of tailoring a microbial or probiotic composition based on the spectrum of bifidobacteria found in the intestine of at least one individual with secretor blood group phenotype but not found in individuals of non-secretor blood group phenotype.

The microbial or probiotic composition of the present invention and the probiotic supplement comprising the composition are particularly suitable and effective, but not limited to in use, for the non-secretor individuals for the enhancement of the diversity of intestinal bifidobacteria. In one embodiment of the present invention, the Bifidobacteria containing supplement contains additionally at least one prebiotic optimised for the growth stimulation or attachment of Bifidobacterium strain or strains. The invention is based on the rationale that as non-secretors were found to have a reduced diversity of Bifidobacteria, that is, they miss certain species, the supplement is particularly enriched with those species missing in non-secretors but found in secretors. As it is known that the non-secretor status and low diversity of Bifidobacteria increase susceptibility to certain diseases (Blackwell, FEMS Microbiology Immunology 1989; 47: 341-350), the increase in the diversity by the supplement as defined in the present invention will have desired effects to the host. A balanced and diverse population of beneficial Bifidobacteria is, therefore, particularly important for non-secretors.

In one embodiment of the invention, the probiotic composition or a supplement comprising the composition is tailored for infants of the non-secretor type.

The present invention relates also to probiotic compositions for use in the treatment and/or prevention of inflammatory bowel disease, diarrhoea, respiratory tract infections, irritable bowel syndrome and/or atopy/allergy.

In one embodiment, the invention is related to probiotic composition for use in the treatment and/or prevention of inflammatory bowel disease (IBD) or symptoms thereof. IBD is an excellent target disease for the invention as not only an altered microbiota composition in the patients has been reported (Sokol et al. Inflamm Bowel Dis. 2006; 12:106-11), but also the therapeutic potential of Bifidobacteria-containing probiotic compositions is known (Macfarlane et al. Clin Rev Clin Lab Sci 2009; 46, 25-54.). Furthermore, it is established (McGovern et al. Hum Molec Genet 2010; 19: 3468-76), that the non-secretor phenotype, i.e. FUT2 gene defect, confers genetic susceptibility to IBD. Hence, it is plausible that the composition according to the present invention is particularly effective in IBD as it aims to modulate the Bifidobacterium composition of non-secretor individuals toward that typically found in secretor individuals. The treatment can be targeted to a relief of the symptoms and/or to prevention of relapses and/or to increasing the overall quality of life in IBD. It also may be administered together with other currently known drugs for IBD. The composition in one embodiment is targeted to those IBD patients with the non-secretor phenotype.

In another embodiment, the invention is related to a probiotic composition for use in the prevention and/or treatment of microbial infections i.e. diarrhoea and respiratory tract infections as also in these indications therapeutic potential of bifidobacteria-containing probiotics (Chouraqui et al. J Pediatr Gastroenterol Nutr. 2004; 38:242-3; de Vrese et al. Clin Nutr. 2005; 24: 479-80) and an increased frequency in non-secretor individuals (Ahmed et al. Infect Immun. 2009; 77:2059-64; Raza et al. BMJ. 1991, 303: 815-8) have been described.

In one embodiment, the invention is related to a probiotic composition for use in the prevention and/or treatment of irritable bowel syndrome (IBS) as decreased levels of bifidobacteria (Mättö et al. FEMS Immunol Med Microbiol. 2005; 43:213-22) and potential of bifidobacteria-containing probiotic products have been described in IBS (Kajander et al. Aliment Pharmacol Ther. 2008; 27:48-57).

In yet another embodiment, the invention is related to probiotic composition for use in the prevention and/or treatment of allergy or atopy in children. It is established that babies who develop allergy have reduced levels of bifidobacteria in their intestine during the first year of life (Björkstén et al. J Allergy Clin Immunol. 2001; 108: 516-20). Moreover, it has been shown that bifidobacteria are detected in breast milk, and the composition of bifidobacterial species in the milk of allergic mothers differs from that of non-allergic mothers (Grönlund et al. Clin Exp Allergy. 2007; 37:1764-72). Products containing Bifidobacteria have shown potential in prevention of atopic eczema (Yoo et al. Proc Am Thorac Soc 2007; 4, 277-282).

In another embodiment, the probiotic composition or a supplement comprising the composition is tailored for infants regardless of their secretor phenotype, whose breast-feeding mother is of the non-secretor blood group type. The probiotic composition or the supplement comprising the composition can be used to enhance the development of a balanced intestinal microbiota composition. Babies of non-secretor mothers are more vulnerable to infections, because the milk of the mother does not contain fucosylated glycans, important for the protection as they bind pathogens. The diet of babies of non-secretor mothers could be supplemented with fucosylated glycans as prebiotics, together with or without the bifidobacterium supplement. The addition of prebiotics to the composition of the present invention is to further augment the efficacy of the probiotic composition by helping the survival of those Bifidobacterium species added into the composition but not commonly found in an individual. A typical prebiotic ingredient is an oligo/polysaccharide which is non-digestible in the upper parts of the oro-gastrointestinal tract. These oligosaccharides include, but are not limited to, fructo-oligosaccharides or inulin, galacto-oligosaccharides, soy oligosaccharides, resistant starch, and polydextrose. An example shown to be particularly suitable for Bifidobacteria is lacto-N-biose I (Kiyohara et al Biosci Biotechol Biochem 2009; 73: 1175-1179). Prebiotics typically are produced by processing from natural sources e.g. from chicory root or milk, alternatively, they may be chemically synthesized. The daily dose needed for a prebiotic effect is typically several grams per day but can vary substantially.

Additionally, in one embodiment the invention is related to probiotics targeted to elderly individuals for supporting the maintence of bifidobacteria diversity and abundance.

The probiotic compositions and supplements so designed may have beneficial effects on the health and/or well-being of a human and may be in the form of, for example, a food product, capsule, tablet or powder. The composition can be formulated into a product of dairy or beverage industry, a functional food product or a nutritional supplement as well as a capsule, emulsion, or powder.

A typical probiotic ingredient is freeze-dried powder containing typically 10¹⁰-10¹² viable probiotic bacterial cells per gram. In addition it normally contains freeze drying carriers such as skim milk, short sugars (oligosaccharides such as sucrose or trehalose). Alternatively, the culture preparation can be encapsulated by using e.g. alginate, starch, xanthan as a carrier. A typical probiotic supplement or capsule preparation contains approximately 10⁹-10¹¹ viable probiotic bacterial cells per capsule as a single strain or multi-strain combination.

A typical probiotic food product, which can be among others fermented milk product, fermented milk-based product or juice, contains approximately 10⁹-10¹¹ viable probiotic bacterial cells per daily dose. Probiotics are incorporated in the product as a probiotic ingredient (frozen pellets or freeze dried powder) or they are cultured in the product, such as yogurt, curd and/or sour milk, during fermentation.

Bifidobacteria containing composition or supplement contains optionally also at least one prebiotic optimised for the growth stimulation of the selected Bifidobacterium strain or strains.

The present invention provides also means for tailoring and/or optimising or potenting an existing probiotic and/or synbiotic product with at least one bifidobacterial strain selected according to the present invention to improve the responsiveness and/or effect of the product in non-secretors.

The present invention also relates to a use of the secretor status of an individual in assessing the need for bifidobacteria-enriched probiotic supplementation. The present invention also relates to a method of assessing the need of an individual for bifidobacteria-enriched probiotic supplementation by determining the secretor status of the individual.

The present invention further relates to a use of the secretor status of an individual in estimating a dose of bifidobacteria supplementation needed for a desired effect. Typically individuals of non-secretor phenotype should need higher doses of probiotics than those with the secretor phenotype.

The present invention also relates to a method of identifying an individual at risk for suffering from a gastrointestinal disorder by determining the secretor status of said individual. The status can be determined, for example, from a sample of saliva, using standard blood grouping methods or from the genomic DNA of an individual by determining adequate mutations in the FUT2 gene (Silva et al. Glycoconjugate Journal 2010; 27: 61-68).

Stabilization of the intestinal bacterial population, especially bifidobacterial population, has been observed to be delayed after severe microbiota disturbances (Mättö et al. 2008). Thus, the present invention provides a use of the secretor status and bifidobacterial species diversity of an individual in following the microbiota stabilisation after such drastic disturbances.

The results of the present invention indicated that non-secretors had a lower diversity of Bifidobacterium in the intestine than secretor individuals. Among strains of Bifidobacterium there were strains that were more common in the intestine of non-secretors. The non-secretors lacked or carried very low or undetectable numbers of several Bifidobacterium genotypes (e.g. genotypes of strains B. adolescentis and B. catenulatum/pseudocatenulatum), which were common in secretors (Table 4). Moreover, B. bifidum, and certain B. adolescentis and B. catenulatum/pseudocatenulatum genotypes, were present more rarely in non-secretor than in secretor individuals (Table 3 and 6). Of the most frequently detected bifidobacterial strains, only B. longum was equally common in both secretor and non-secretor individuals. Accordingly, some bifidobacteria are present in GI tract of almost all humans, but non-secretors miss some or many of the bifidobacterial strains i.e. all human share some bifidobacterial species, but non-secretors miss many bifidobacterial species present commonly in secretors. Based on these findings, the probiotic composition and/or supplement of the present invention contain in particular those bifidobacterial species or strains abundant in individuals with the secretor phenotype.

The invention will be described in more detail by means of the following examples. The examples are not to be construed to limit the claims in any manner whatsoever.

### EXAMPLES

### Materials and Methods

The materials and methods described herein are common to examples 1 to 7.

59 healthy adult volunteers (52 females and 7 males) we recruited to the study. Both faecal and blood samples were collected from 59 volunteers. The age of the volunteers ranged from 31 to 61 and was in average 45 years.

Faecal samples were frozen within 5 hours from defecation. DNA from 0.3 g of faecal material was extracted by using the FASTDNA® SPIN KIT FOR SOIL (Qbiogene). Partial bifidobacterial 16S rRNA gene was amplified by PCR with bifidobacterial specific primers Bif164F and Bif662R+GC (Satokari et al., Appl Environm Microbiol 2001, 67, 504-513). The specificity of the primers was tested with Bifidobacterium strains (B. adolescentis E-981074, B. bifidum E-97795, B. lactis E-97847, B. longum E-96666, B. angulatum DSM 20098 and Bifidobacterium catenulatum DSM 16992), which are the most common Bifidobacterium species inhabiting human gut, as well as 43 other bacterial strains having representatives of common human cut bacteria. Amplified PCR fragments were separated in 8% DGGE gel with denaturing gradient from 45% to 60%. DGGE gels were run at 70 V for 960 mins. DGGE gels were stained with SYRBSafe for 30 mins and documented with Safelmager Bluelight table (Invitrogen) and Aplhalmager HP (Kodak) imaging system.

Digitalised DGGE gel images were imported to the Bionumerics-program version 5.0 (Applied Maths) for normalisation and band detection. Bands were normalised with marker samples constructed from bifidobacterial strains. Band search and bandmatching was performed as implemented in Bionumerics. Bands and bandmatching were manually checked and corrected.

The bands were excised from bifidobacteria-DGGE gels. DNA from bands was eluted by incubating bands in 50 μl sterile H₂O at +4° C. overnight. The correct position and purity of only each of the excised bands were tested by amplifying DNA in bands and running the amplified fragments along the original samples in DGGE. Bands, which only produced single bands and were in the correct position in the gels, were sequenced in Eurofins MWG (Germany). The sequences were trimmed, manually checked and corrected for ambiguous bases and aligned by ClustalW. The closest relatives of the sequences were searched using Blast and NCBI nr database. Distance matrix of the aligned sequences was used to compare the similarity of the sequences.

### Example 1

Secretor status was determined from the blood samples using the standard in-house blood grouping protocols of Finnish Red Cross Blood Service. Secretor status was determined from 59 individual and 48 were secretors and seven were non-secretors. Secretor status of 4 samples could not be determined.

### Example 2

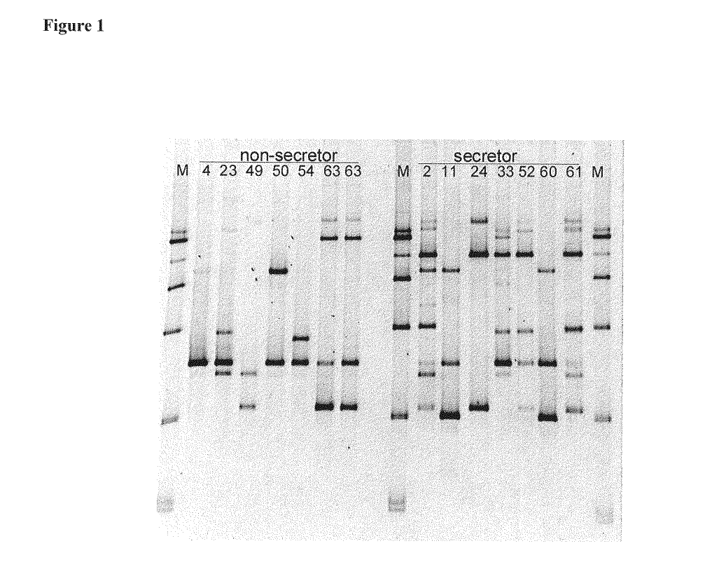

DGGE analysis targeted for the faecal bifidobacterial population was performed as described above in the material and methods. DGGE gel images showed fewer numbers of bands in the samples obtained from the non-secretor individuals than in the samples from secretor individuals, indicating that fewer bifidobacterial genotypes were present in non-secretor than in secretor individuals. In average, non-secretors had 2.5 (maximum 4) bands and secretors 5.2 bands (maximum 11 bands) in bifidobacterial DGGE profiles. In five samples bifidobacteria were not detected (one non-secretor sample and 4 secretor samples). The Bifidobacterial profiles of all non-secretor individuals and selected bifidobacterial profiles of the secretor individuals are presented in FIG. 1.

### Example 3

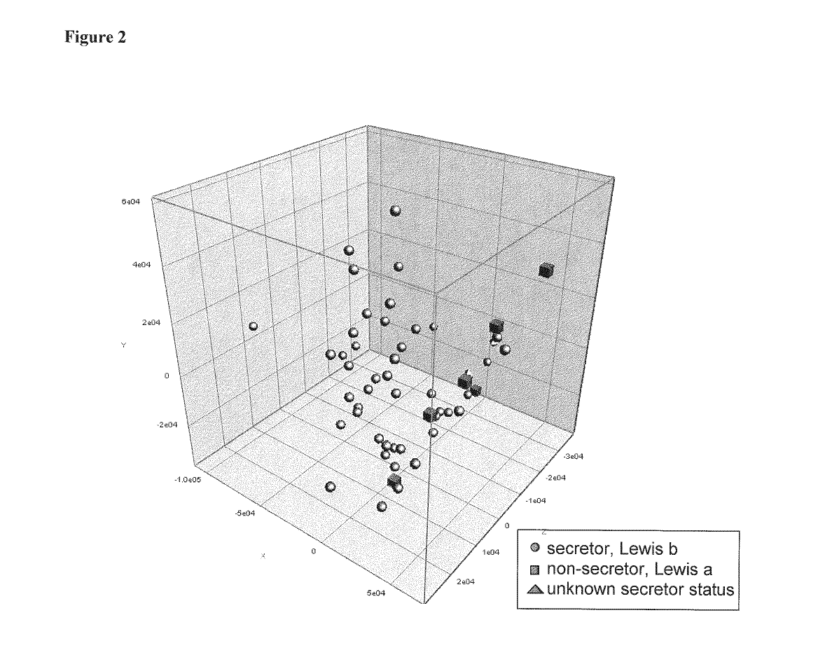

DGGE analysis targeted for the faecal bifidobacterial population was performed as described above. Principal component analysis (PCA) was performed as implemented in the Bionumerics software package. PCA based on intensities of bands detected by DGGE, was used to ordinate samples and to find out the bands which predominantly contributed to the principal components. Images of DGGE gels were analysed using the Bionumerics to allow statistical analysis between samples. PCA based on intensities of bands in DGGE gels showed grouping of the samples obtained from the non-secretors. The first and second principal component explained of the 56.3% of the total variance. The results are presented in FIG. 2.

### Example 4

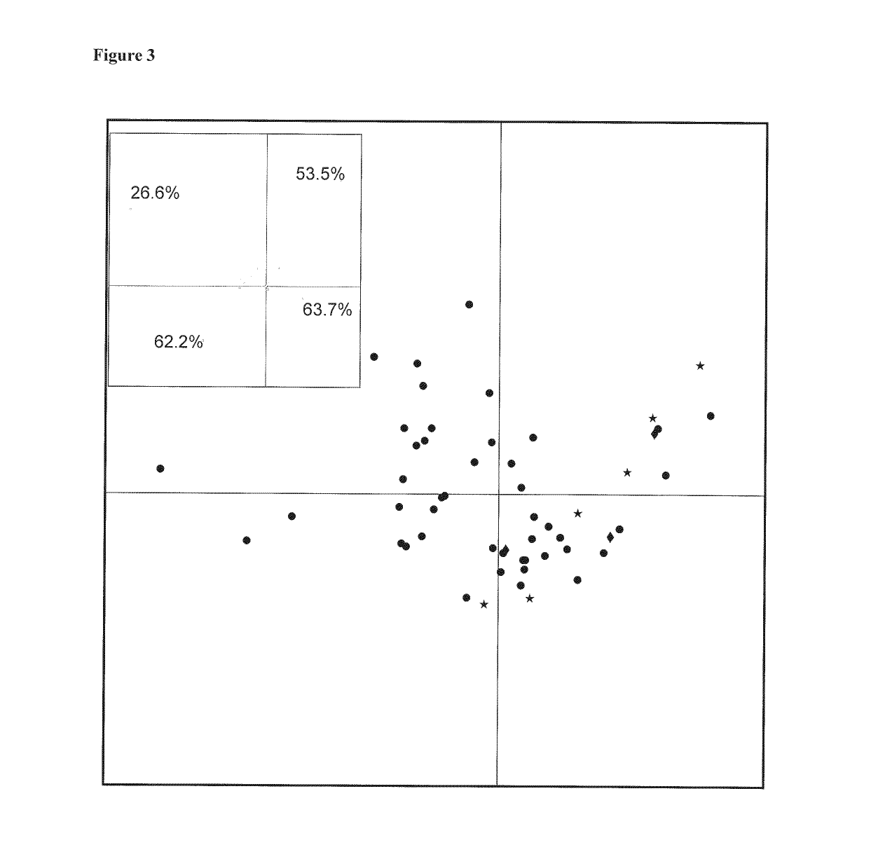

DGGE analysis targeted to the faecal bifidobacterial population was performed as described above. PCA based on intensities of bands detected by bifidobacterial DGGE was used to ordinate samples and to find out the bands which most contributed to the principal components. In the PCA biplot, the first and second principal component contributed 56.3% of the total variance. The bands in positions 26.6%, 53.3%, 62.2% and 63.7% contributed most clearly to the components. These bands were the most commonly detected bands in the samples (Table 5). The PCA biplot based on bifidobacterial DGGE profiles is presented in FIG. 3.

### Example 5

DGGE analysis targeted for the faecal bifidobacterial population was performed as described above. The Shannon diversity index based on band intensities was used to summarise the diversity of bifidobacteria in the samples. The index calculations and t-tests were done. The Shannon index, which describes diversity based on abundance and eveness of species, showed that bifidobacterial diversity was statistically significantly reduced in non-secretor individuals in comparison to secretor individuals (p=0.009).

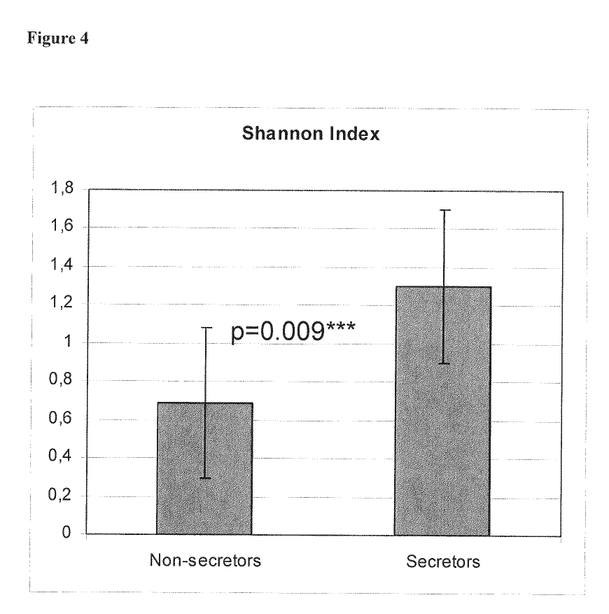

Thus, non-secretor individuals have lower bifidobacterial diversity than secretor individuals. The results are presented in FIG. 4.

### Example 6

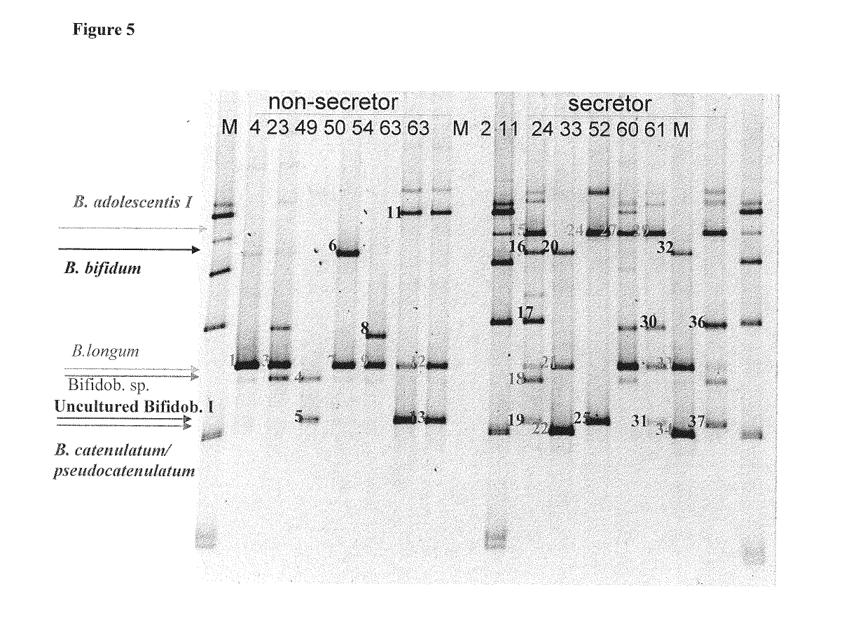

DGGE analysis and identification of the bands by sequencing was performed as described above. Identification was based on the Blast search of the sequences obtained from the excised bands of the DGGE gels. The results showed that several common bifidobacterial genotypes were missing or were present rarely in non-secretor individuals as compared to those found in secretor individuals. Specifically, most commonly detected genotypes of B. adolescentis (bands 15, 24, 27, and 29 in FIG. 5) and B. catenulatum/pseudocatenulatum (bands 22 and 34 in FIG. 5) and genotypes related to uncultured Bifidobacterium (bands 5, 13, 19, 25, 31, and 37 in FIG. 5), or those spieces and/or strains of Bifidobacterium whose detailed identification at the spieces-level requires further analyses, e.g. sequencing, were not detected in non-secretors. Moreover, genotypes related to B. bifidum (bands 6, 8, 11, 16, 17, 20, 30, 32, and 36 in FIG. 5) and uncultured Bifidobacterium were more rarely detected in non-secretor individuals than in secretor individuals. The most commonly detected Bifidobacterium genotypes in the entire set of study samples were also those whose occurrence differed between the non-secretor individuals and secretor individuals (bold in Table 5), except for B.

longum which was equally common in both secretor individuals and non-secretor individuals. Thus, the results indicated that non-secretors lacked or carried a low number of several Bifidobacterium genotypes, which were common in secretors. The results are presented in FIG. 5 and Table 5.

### Example 7

DGGE analysis and band position analysis using the BioNumerics-software were performed as described above. The result showed that the Bifidobacterium genotypes present in the nonsecretor individuals represented Bifidobacterium genotype 4 (band position 16.3%), Bifidobacterium genotype 6 (band position 20.4%), Bifidobacterium genotype 7 (band position 22.3%), Bifidobacterium bifidum (band position 29.7%), Bifidobacterium genotype 12 (band position 43.8%), Bifidobacterium genotype 16 (band position 47.3%), Bifidobacterium genotype 17 (band position 49.5%), Bifidobacterium genotype 18 (band position 55.0%), Bifidobacterium genotype 20 (band position 62.2%) and Bifidobacterium longum (band position 53.5%). (Table 5, FIG. 6).

TABLE 5
The identification of the band positions and the detection frequency of bands
in non-secretors (NSS, n = 6) and secretors (SS, n = 42). The band
positions, which frequency differed between non-secretors and secretors are in bold.

| | Number of | | Total number | | | |
|---|---|---|---|---|---|---|
| Closest relative by Blast (closest | Sequenced | Band | of bands | % in | % in | % in |
| cultured relative, similarity in Blast) | bands | position | detected | NSS | SS | all |
| B. longum | 7 | 53.5% | 47 | 83 | 88 | 87 |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | | | | | | |
| [symbol] | | [symbol] | [symbol] | [symbol] | [symbol] | [symbol] |
| [symbol] | [symbol] | [symbol] | [symbol] | [symbol] | [symbol] | [symbol] |
| [symbol] | | | | | | |
| [symbol] | | [symbol] | [symbol] | [symbol] | [symbol] | [symbol] |
| [symbol] | [symbol] | [symbol] | [symbol] | [symbol] | [symbol] | [symbol] |
| B. adolescentis b | 1 | 22.3% | 10 | 17 | 19 | 19 |
| Bifidobacterium sp. | | 47.3% | 9 | 17 | 17 | 17 |
| B. catenulatum, 477/479, 99%) | | | | | | |
| uncultured Bifidobacterium | | 43.8% | 8 | 17 | 15 | 15 |
| (B. adolescentis 476/481, 98%,/ | | | | | | |
| B. ruminantium 455/457, 99%)* | | | | | | |
| uncultured Bifidobacterium | | 55.0% | 7 | 17 | 13 | 13 |
| (B. adolescentis 465/468, 99%) | | | | | | |
| [symbol] | | [symbol] | [symbol] | [symbol] | [symbol] | [symbol] |
| [symbol] | | | | | | |
| nd | | 16.3% | 5 | 17 | 8 | 9 |
| nd | | [symbol] | [symbol] | [symbol] | [symbol] | [symbol] |
| Other (11 band positions) | | | 1 . . . 4 | 0-17% | 2-8% | 2-7% |

*The sequences of two bands classified in to this band position (43.8%) were not identical and they had similarity 97.3%

### Example 8

In this example, the number of volunteers was increased to 71 by recruiting 12 new volunteers in addition to the 59 volunteers of the previous Examples 1-7. For these 71 samples, in addition to phenotyping, secretor status was genotyped by sequencing the coding exon of FUT2 as described in Silva et al. (Silva et al. Glycoconj J 2010; 27: 61-68) and Ferrer-Admetlla et al. (Mol Biol Evol 2009; 26: 1993-2003). Genotyping of FUT2 exon enabled determination of secretor status for Lewis-negative individuals, whose phenotypic secretor status could not be determined. The DGGE analysis of the faecal bifidobacterial genotypes and data-analysis were performed as described above, except that in PCA, the presence and absence of bands was applied. Statistical analysis, Anova, was computed with statistical programming language R, version 2.10.1.

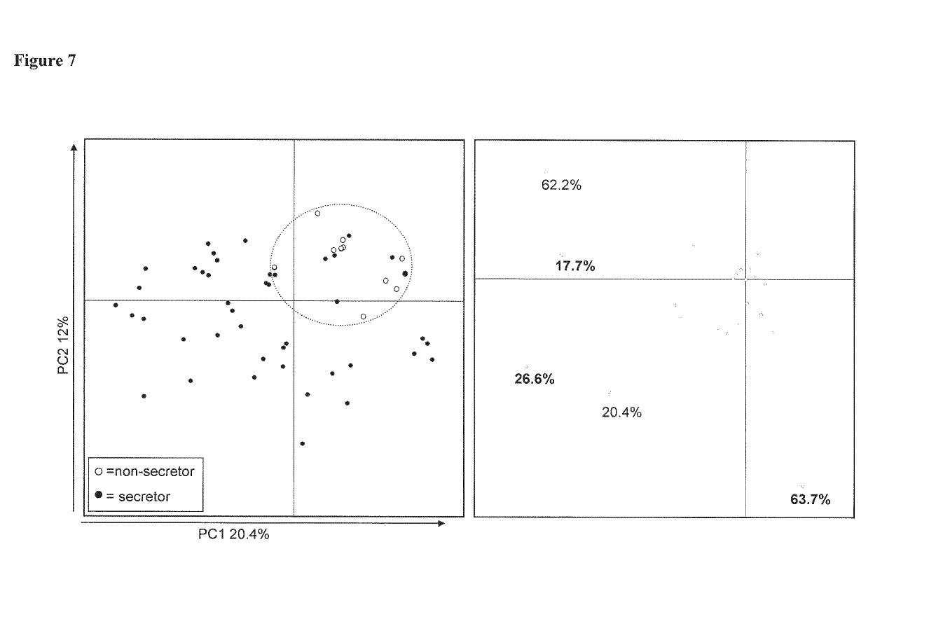

In the enlarged dataset (n=71), 57 individuals had the secretor phenotype and 14 were non-secretors. Similarly to the PCA results in Examples 3 and 4, the results of non-secretor individuals formed a separate cluster within the secretor individuals in the PCA analysis of bifidobacterial DGGE profiles. The PCA biplot based on bifidobacterial DGGE profiles is presented in FIG. 7. The observed clustering in PCA indicated that bifidobacterial population was altered in the samples from non-secretor individuals in comparison to the those from the secretor individuals. These results confirmed the result with a smaller number of samples described in Examples 3 and 4.

The band positions that mostly contributed to the PCA clustering were 17.7%, 20.4%, 26.6%, 62.2% and 63.7% (FIG. 7). These band positions were also among the most commonly detected in the samples from the secretor individuals (Table 6). Band positions 17.7% (B. adolescentis) and 63.7% (B. catenulatum/pseudocatenulatum) were absent from the non-secretor individuals. All other common band positions (present in more than 10% of the samples), except band position 53.5% related to B. longum, were clearly less frequent in the non-secretor individuals than in the secretor individuals (Table 6). The band positions associated to the secretor status were related to B. adolescentis, B. catenulatum/pseudocatenulatum, B. dentium and B. bifidum. Thus, the results indicated that non-secretors lacked or had rarely several of the Bifidobacterium genotypes that were common in the secretor samples.

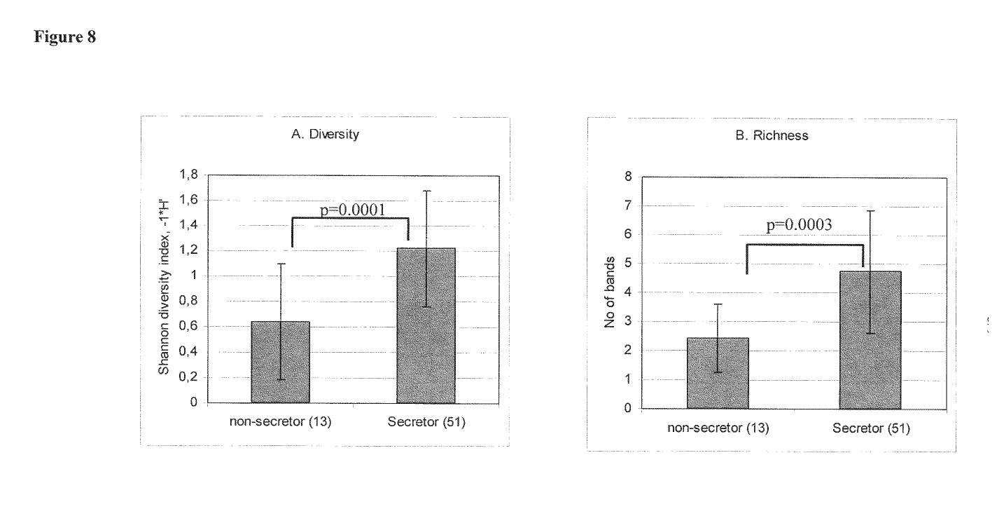

Similarly to Example 5, the Shannon diversity index based on band intensities was used to demonstrate diversity of bifidobacteria in the samples. In addition, the number of bands was used to demonstrate the richness of bifidobacterial species. The Shannon diversity index and the number of bands showed that bifidobacterial diversity and richness was statistically significantly reduced in the non-secretor individuals in comparison to the secretor individuals (p=0.0001 and 0.0003, respectively). In average, the number of bands per sample in non-secretors was almost two times (1.9) higher than that in secretors. In average, non-secretor individuals had 2.5 (maximum 5) bands and secretor individuals 4.7 bands (maximum 11 bands) in bifidobacterial DGGE profile. Thus, non-secretor individuals had a lower bifidobacterial diversity and richness than secretor individuals. The results are presented in FIG. 8.

TABLE 6
Identification of the band positions of Bifidobacterial
DGGE by sequencing and the detection frequencies of the
bands in secretor (14) and non-secretor individuals (57).

| | Band | | % in | % in |
|---|---|---|---|---|
| | posi- | | non- | secre- |
| Identification by sequencing* | tion | Hits | secretors | tors |
| B. longum | 53.5% | 56 | 79 | 79 |
| B. adolescentis | 62.2% | 41 | 50 | 60 |
| B. adolescentis | 26.6% | 40 | 14 | 67 |
| | 17.7% | 18 | 0 | 32 |
| B. catenulatum/pseudocatenulatum | 63.7% | 18 | 0 | 32 |
| B. bifidum | 29.7% | 17 | 7 | 28 |
| | 20.4% | 16 | 7 | 26 |
| B. adolescentis | 22.3% | 13 | 14 | 19 |
| B. catenulatum/pseudocatenulatum | 43.8% | 13 | 14 | 19 |
| B. catenulatum/pseudocatenulatum | 47.3% | 9 | 7 | 14 |
| B. adolescentis | 55.0% | 9 | 7 | 14 |
| B. catenulatum/pseudocatenulatum | 44.5% | 8 | 7 | 12 |
| | 39.3% | 7 | 7 | 11 |
| | 16.3% | 5 | 7 | 7 |
| | 46.1% | 5 | 0 | 9 |
| | 24.9% | 4 | 7 | 5 |
| | 3.5% | 3 | 0 | 5 |
| B. adolescentis | 45.7% | 4 | 0 | 7 |
| | 12.6% | 3 | 0 | 5 |
| | 33.0% | 3 | 0 | 5 |
| | 49.5% | 3 | 7 | 4 |
| | 69.3% | 3 | 7 | 4 |
| | 7.5% | 1 | 0 | 2 |
| | 36.4% | 1 | 0 | 2 |
| | 57.3% | 1 | 0 | 2 |
| | 64.8% | 1 | 0 | 2 |

*The similarity of the partial 16S rDNA sequence (475-490 bp) to the type strain of the indicated species was ≧98%.

### Example 9

Quantitative PCR (qPCR) method was applied to detect and quantify the 16S rRNA gene copies of bacteria, bifidobacteria and 4 bifidobacterial groups/species, B. bifidum, B. longum group, B. catenulatum/pseudocatenulatum and B. adolescentis in faecal samples of non-secretor individuals (n=14) and secretor individuals (n=57). The primers and annealing temperature for each primer pair are shown in Table 7. For each primer pair, a reaction mixture of 25 μl was composed of 0.3 μM of each primer (Sigma-Aldrich, UK), 1×Power SYBR Green PCR Master Mix (Applied Biosystems, CA, USA), 4 μl faecal DNA diluted to the concentration of 1 ng/μl for bifidobacterial group-specific primers and to the concentration of 0.1 ng/μl for universal and bifidobacterial primers. The amplification conditions in ABI Prism 7000 instrument (Applied Biosystems, CA, USA) were one cycle of 95° C. for 10 mins, followed by 40 cycles of 95° C. for 15 s, and appropriate annealing temperature (Table 6) for 60 s. Melting temperature curves from 60° C. to 95° C. were analysed to determine the specificity of the amplification. All the samples and standards were analysed in three replicates. Standard curves for each bacterial group were constructed from the corresponding bacterial strain (Table 7) by using 10-fold dilutions of known concentrations of bacterial genomic DNA (from 10 ng/μl to 0.0004 ng/μl). Genomic DNA from standard strains was extracted by QIAmp®DNA mini kit (Qiagen) combined with cell lysis in the FastPrep® Instrument (MP Biomedicals, CA, USA). GenEx Enterprise v.5.2.6.34 (MultiD Analyses AB, Sweden) was used for analysis of standard curves and reverse quantification of the samples. The statistical analysis, Wilcoxon test, was computed with statistical programming language R, version 2.10.1.

TABLE 7
The primers targeting the 16S rRNA gene, annealing temperature
and the strains used as standards for qPCR.

| | | | Anneali | |
|---|---|---|---|---|
| | | | neal- | |
| Group | Primer | Primer sequence 5′→3′ | ing-T | Standard strain |
| Bifidobacteria | qBifF | TCGCGTCYGGTGTGAAAG | 59 | B. bifidum E-97795 |
| | qBifR² | CCACATCCAGCRTCCAC | | (NCIMB41171) |
| B. longum group* | BiLON-1 | CAGTTGATCGCATGGTCTT | 62 | B. longum E-96664 |
| | BiLON-2³ | TACCCGTCGAAGCCAC | | (ATCC15707) |
| B. bifidum | BiBIF-1 | CCACATGATCGCATGTGATTG | 58 | B. bifidum |
| | BiBIF-2³ | CCGAAGGCTTGCTCCCAAA | | E-97795 |
| | | | | (NCIMB41171) |
| B. catenulatum/ | BiCATg-1 | CGGATGCTCCGACTCCT | 62 | B. catenulatum |
| pseudocatenula- | BiCATg-2³ | CGAAGGCTTGCTCCCGAT | | DSM 16992 |
| tum | | | | |
| B. adolescentis | BiADOg-1a | CTCCAGTTGGATGCATGTC | 58 | B. adolescentis |
| group | BiADOg-1b | TCCAGTTGACCGCATGGT | | E-981074 |
| | BiADO-2^{3,**} | CGAAGGCTTGCTCCCAGT | | (ATCC15703) |

*Amplifies species B. longum, B. infantis, B. suis.
**Two forward primers to amplify B. adolescentis genotypes A and B. References:
¹Tseng et al. Clin Chem 2003; 49: 306-309.
²Rinttila et al. J Appl Microbiol 2004; 97: 1166-1177.
³Matsuki et al. Appl Environ Microbiol 2004; 70: 167-173.

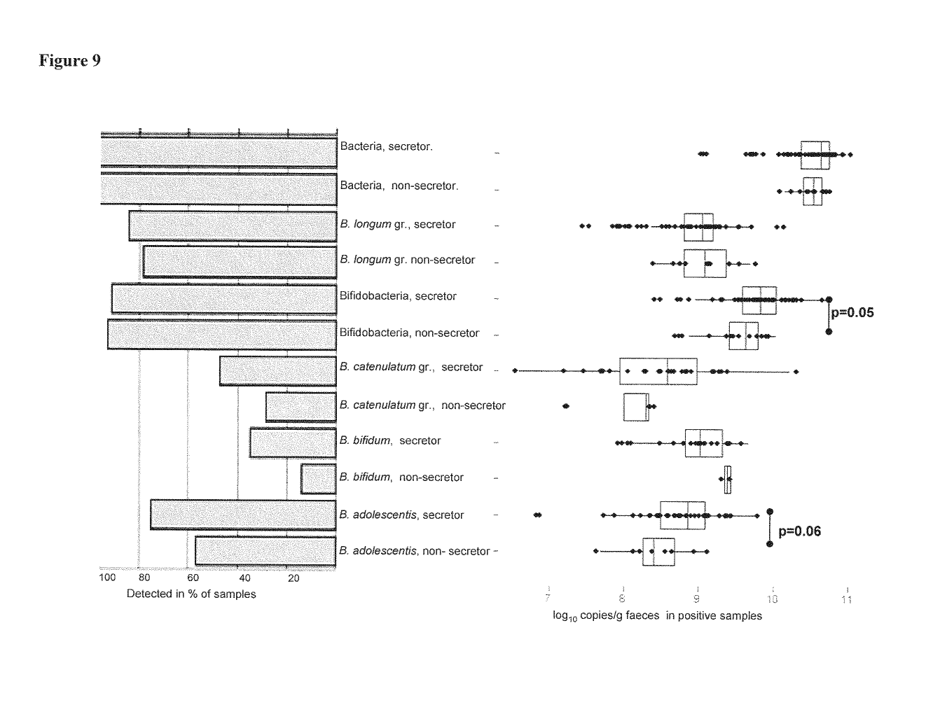

Bifidobacteria were detectable in over 90% of the samples in samples from both non-secretor and secretor individuals (FIG. 9). The total amount of bifidobacteria was lower (p=0.05) and a fewer number of bifidobacterial species were present in the samples of the non-secretor individuals in comparison to those of the secretor individuals (FIG. 9). All bifidobacterial groups were detected less frequently in non-secretor than in secretor samples, hence confirming the DGGE results. B. bifidum was detected in 14% of the non-secretor samples (as compared to 35% of the secretor samples), B. catenulatum/pseudocatenulatum in 29% of the non-secretor samples (as compared to 47% of the secretor samples) and B. adolescentis in 57% of the non-secretor samples (as compared to 75% of the secretor samples). Moreover, in samples with detectable amounts of B. adolescentis group, the abundance of the B. adolescentis was lower in the non-secretor than in secretor samples (p=0.055) (FIG. 9).

### Example 10

We isolated bifidobacterial strains from samples of the non-secretor and secretor individuals and analysed their 16S rRNA gene fragments in the DGGE gel along with faecal samples in order to identify the strains corresponding the observed DGGE band positions (i.e. DGGE genotypes). Strains were isolated by applying the TNO TIM-1 model, which mimics conditions in the stomach and small intestine. Faecal slurries were prepared from pooled non-secretor samples (total 12.1 g of faeces), two individual samples of non-secretors (1.9 and 2 g of faeces) and pooled secretor samples (total 9.8 g of faeces). The same faecal samples were used in the DGGE analysis and strain isolation. Faecal slurries for TIM-1 acquired by mixing faeces with artificial saliva and sterile water/milk were used as input for the TIM-1 model. Two states of the model were applied: In one state, T1/2 for emptying the gastric content was set to 20 min, pH change from pH 2.0 to 1.7 in 30 min and level of gastric secretion on 20%. In another state, gastric emptying halftime was set 30 min, gastric pH decrease from 5.0 to 1.8 in 90 min and level of gastric secretion to 100° A. The gastric content was passed into the duodenal compartment, where it was neutralized to pH 6.4, and bile and pancreatin were added, followed passage (time 10 minutes) into the jejunum compartment and into the ileum compartment. In each compartment the physiological concentrations of bile salts, pancreatic enzymes and electrolytes simulated in combination with an average physiological passage through the small intestine. The samples were collected from after 120-180, 180-240 and 240-300 mins treatment in model and bifidobacterial isolated using beerens and RB (raffinose bifidobacterial media) media. The isolates were incubated in anaerobic conditions for 72 hours at 37° C. The strains from faecal samples of secretor individuals were also isolated from faecal slurries by plating directly on beerens agar and incubating in anaerobic conditions for 72 hours at 37° C. The isolates were screened with RAPD using primers OPA-2 as described in Mättö et al. (J Appl Microbiol 2004; 98: 459-470). The strains representing different RAPD profiles were deposited to a local strain collection and analysed by DGGE to find the band positions they corresponded to. Genomic DNA from strains was extracted by QIAmp®DNA mini kit (Qiagen) combined with cell lysis in the FastPrep®Instrument (MP Biomedicals, CA, USA). The strains were identified by sequencing of the 16S rRNA gene fragment of approximately 700 base pair in length. The DGGE analysis was performed as described above.

A total of 274 Bifidobacterial strains from non-secretor individuals and 360 strains from secretor individuals were isolated. The isolates were screened using RAPD for detection of different bifidobacterial strains. In RAPD screening of isolates, 15 different bifidobacterial isolates with different RAPD profiles originated from samples from non-secretor individuals and 28 bifidobacterial isolates originated from samples from secretor individuals.

The strains accounted for 15 of the 26 band positions (DGGE genotypes) detected in the DGGE profiles of the faecal samples. Moreover, the strains accounted for almost all common bifidobacterial DGGE genotypes (12 of the 13 strains present in more than 10% of samples) (Table 6). Strains from non-secretor individuals were found for 6 DGGE genotypes, whereas strains from secretor individuals were found for 13 DGGE genotypes. The band positions/DGGE genotypes, DNA sequences and the corresponding strains are listed in Table 8. Some strains had several 16S rRNA copies and thus one strain may correspond to several band positions.

TABLE 8
The sequences and identification of bifidobacterial band positions in DGGE and the presence
of corresponding strains from secretor and non-secretor individuals. The sequence for the
16S rRNA gene fragment, that is amplified and analysed in DGGE with primers Bif164F and
Bif662R, is showed for each position. Bifidobacterial genotype number refers to the
genotype in Table 1 and 2.

| | Strains | Strains | | |
|---|---|---|---|---|
| Band | from | from | | |
| po- | se- | non-se- | Identification | |
| sition | cretors | cretors | of the strains | Sequence 5′->3′ |
| 16.3% | + | + | B. adolescentis | no sequence. Strains having band in this position have |
| | | | (genotype 4) | always another band in position 62.2%. |
| 17.7% | + | − | B. adolescentis | no sequence. Strains having band in this position have |
| | | | (genotype 5) | always another band in position 26.6% and in 62.2% |
| 20.4% | + | − | B. adolescentis | no sequence Strains having band in this position have |
| | | | (genotype 6) | always another band in position 26.6.2% |
| 22.3% | + | + | B. adolescentis | CTCCAGTTGGATGCATGTCCTTCTGGGAAAGATTCTATCGGTATGGGATGGGGTCGCGTCCTA |
| | | | (genotype 7) | TCAGCTTGATGGCGGGGTAACGGCCCACCATGGCTTCGACGGGTAGCCGGCCTGAGAGGGC |
| | | | | GACCGGCCACATTGGGACTGAGATACGGCCCAGACTCCTACGGGAGGCAGCAGTGGGGAAT |
| | | | | ATTGCACAATGGGCGCAAGCCTGATGCAGCGACGCCGCGTGCGGGATGACGGCCTTCGGGT |
| | | | | TGTAAACCG |
| | | | | (SEQ ID NO: 1) |
| 24.9% | + | − | B. catenulatum/ | no sequence Strains having band in this position have |
| | | | pseudocatenulatum | always another band in position 44.5% |
| | | | (genotype 8) | |
| 26.6% | + | − | B. adolescentis | CTCCAGTTGGATGCATGTCCTTCTGGGAAAGATTCATCGGTATGGGATGGGGTCGCGTCCTAT |
| | | | | CAGCTTGATGGCGGGGTAACGGCCCACCATGGCTTCGACGGGTAGCCGGCCTGAGAGGGCG |
| | | | | ACCGGCCACATTGGGACTGAGATACGGCCCAGACTCCTACGGGAGGCAGCAGTGGGGAATA |
| | | | | TTGCACAATGGGCGCAAGCCTGATGCAGCGACGCCGCGTGCGGGATGACGGCCTTCGGGTT |
| | | | | GTAAACCGC |
| | | | | (SEQ ID NO: 2) |
| 29.7% | + | − | B. bifidum | TTCCACATGATCGCATGTGATTGTGGGAAAGATTCTATCGGCGTGGGATGGGGTCGCGTCCT |
| | | | | ATCAGCTTGTTGGTGAGGTAACGGCTCACCAAGGCTTCGACGGGTAGCCGGCCTGAGAGGG |
| | | | | CGACCGGCCACATTGGGACTGAGATACGGCCCAGACTCCTACGGGAGGCAGCAGTGGGGAA |
| | | | | TATTGCACAATGGGCGCAAGCCTGATGCAGCGACGCCGCGTGAGGGATGGAGGCCTTCGGG |
| | | | | TTGTAAACCT |
| | | | | (SEQ ID NO: 3) |
| 39.3% | + | − | B. dentium | CTCCGGTTGGATGCATGTCCTTCCGGGAAAGATTCCATCGGTATGGGATGGGGTCGCGTCCT |
| | | | (genotype 11) | ATCAGCTTGATGGCGGGGTAACGGCCCACCATGGCTTCGACGGGTAGCCGGCCTGAGAGGG |
| | | | | CGACCGGCCACATTGGGACTGAGATACGGCCCAGACTCCTACGGGAGGCAGCAGTGGGGAA |
| | | | | TATTGCACAATGGGCGCAAGCCTGATGCAGCGACGCCGCGTGCGGGATGGAGGCCTTCGGG |
| | | | | TTGTAAACCGC |
| | | | | (SEQ ID NO: 4) |
| 43.8% | + | − | B. catenulatum/ | CTCCGACATGACGCATGTCGTGTTGGGAAAGATTCATCGGTATGGGATGGGGTCGCGTCCTA |
| | | | pseudocatenulatum | TCAGGTAGTCGGCGGGGTAACGGCCCACCGAGCCTACGACGGGTAGCCGGCCTGAGAGGG |
| | | | (genotype 12) | CGACCGGCCACATTGGGACTGAGATACGGCCCAGACTCCTACGGGAGGCAGCAGTGGGGAA |
| | | | | TATTGCACAATGGGCGCAAGCCTGATGCAGCGACGCCGCGTGCGGGATGACGGCCTTCGGG |
| | | | | TTGTAAACCGC |
| | | | | (SEQ ID NO: 5) |
| 44.5% | + | − | B. catenulatum/ | CTCCAACATGACGCATGTNGTGTCGGGAAAGATTCATCGGTATGGGATGGGGTCGCGTCCTA |
| | | | pseudocatenulatum | TCAGGTAGTCGGCGGGGTAACGGCCCACCGAGCCTACGACGGGTAGCCGGCCTGAGAGGG |
| | | | (genotype 13) | CGACCGGCCACATTGGGACTGAGATACGGCCCAGACTCCTACGGGAGGCAGCAGTGGGGAA |
| | | | | TATTGCACAATGGGCGCAAGCCTGATGCAGCGACGCCGCGTGCGGGATGACGGCCTTCGGG |
| | | | | TTGTAAACCGC |
| | | | | (SEQ ID NO: 6) |
| 45.7% | + | − | B. adolescentis | CTCCAGTTGACCGCATGGTCCTCTGGGAAAGATTCATCGGTATGGGATGGGGTCGCGTCCTA |
| | | | (genotype 14) | TCAGCTTGATGGCGGGGTAACGGCCCACCATGGCTTCGACGGGTAGCCGGCCTGAGAGGGC |
| | | | | GACCGGCCACATTGGGACTGAGATACGGCCCAGACTCCTACGGGAGGCAGCAGTGGGGAAT |
| | | | | ATTGCACAATGGGCGCAAGCCTGATGCAGCGACGCCGCGTGCGGGATGACGGCCTTCGGGT |
| | | | | TGTAAACCGC |
| | | | | (SEQ ID NO: 11) |
| 47.3% | − | + | B. catenulatum/ | CTCCGACTCCTCGCATGGGGTGTCGGGAAAGATTATATCGGTATGGGATGGGGTCGCGTCCT |
| | | | pseudocatenulatum | ATCAGGTAGTCGGCGGGGTAACGGCCCACCGAGCCTACGACGGGTAGCCGGCCTGAGAGG |
| | | | (genotype 16) | GCGACCGGCCACATTGGGACTGAGATACGGCCCAGACTCCTACGGGAGGCAGCAGTGGGGA |
| | | | | ATATTGCACAATGGGCGCAAGCCTGATGCAGCGACGCCGCGTGCGGGATGACGGCCTTCGG |
| | | | | GTTGTAAACCG |
| | | | | (SEQ ID NO: 7) |
| 53.5% | + | + | B. longum | CTCCAGTTGATCGCATGGTCTTCTGGGAAAGCTTTCGCGGTATGGGATGGGGTCGCGTCCTA |
| | | | | TCAGCTTGACGGCGGGGTAACGGCCCACCGTGGCTTCGACGGGTAGCCGGCCTGAGAGGGC |
| | | | | GACCGGCCACATTGGGACTGAGATACGGCCCAGACTCCTACGGGAGGCAGCAGTGGGGAAT |
| | | | | ATTGCACAATGGGCGCAAGCCTGATGCAGCGACGCCGCGTGAGGGATGGAGGCCTTCGGGT |
| | | | | TGTAAACCTC |
| | | | | (SEQ ID NO: 8) |
| 55.0% | − | − | B. adolescentis | CTCCGACATGACGCATGTCGTGTCGGGAAAGATTCATCGGTATGGGATGGGGTCGCGTCCTA |
| | | | (genotype 18) | TCAGCTTGATGGCGGGGTAACGGCCCACCATGGCTTCGACGGGTAGCCGGCCTGAGAGGGC |
| | | | | GACCGGCCACATTGGGACTGAGATACGGCCCAGACTCCTACGGGAGGCAGCAGTGGGGAAT |
| | | | | ATTGCACAATGGGCGCAAGCCTGATGCAGCGACGCCGCGTGCGGGATGACGGCCTTCGGGT |
| | | | | TGTAAACCGC |
| | | | | (SEQ ID NO: 9) |
| 62.2% | + | + | B. adolescentis | CTCCAGTTGACCGCATGGTCCTCTGGGAAAGCTTTTGCGGTATGGGATGGGGTCGCGTCCTA |
| | | | (genotype 20) | TCAGCTTGATGGCGGGGTAACGGCCCACCATGGCTTCGACGGGTAGCCGGCCTGAGAGGGC |
| | | | | GACCGGCCACATTGGGACTGAGATACGGCCCAGACTCCTACGGGAGGCAGCAGTGGGGAAT |
| | | | | ATTGCACAATGGGCGCAAGCCTGATGCAGCGACGCCGCGTGCGGGATGACGGCCTTCGGGT |
| | | | | TGTAAACCGC |
| | | | | (SEQ ID NO: 10) |
| 63.7% | + | + | B. catenulatum/ | TGCGGGATGACGGCCTTCGGGTTGTAAACCGCTTTTGATCGGGAGCAAGCCTTCGGGTGAGT |
| | | | pseudocatenulatum | GTACCTTTCGAATAAGCACCGGCTAACTACGTGCCAGCAGCCGCGGTAATACGTAGGGTGCA |
| | | | | AGCGTTATCCGGAATTATTGGGCGTAAAGGGCTCGTAGGCGGTTCGTCGCGTCCGGTGTGAA |
| | | | | AGTCCATCGCTTAACGGTGGATCTGCGCCGGGTACGGGCGGGCTGGAGTGCGGTAGGGGAG |
| | | | | ACTGGAAT |
| | | | | (SEQ ID NO: 12) |

### Example 11

The survival of bifidobacteria in TNO TIM-1 model treatment was analysed by counting of viable bacteria before and after the treatment as described in Example 10. The bifidobacterial survival in the TNO TIM-1 model was analysed from faecal slurries, prepared from pooled non-secretor samples (total 12.1 g faeces) and secretor samples (total 9.8 g of faeces). Samples were collected from faecal slurries before the TIM-1 treatment (intake samples), after 120-180 mins, 180-240 mins, and 240-300 mins of treatment. Dilution series of collected samples were plated in duplicate on beerens and RB media and incubated for 72 hours at 37° C.

The survival of bifidobacteria from secretor pool was 2.4 times higher on RB agar or 32 times higher on Beerens agar than the survival of bifidobacteria from non-secretor pool (Table 9). These results indicated that cultivable bifidobacterial population in non-secretor individuals differed from bifidobacterial population in secretor individuals. Furthermore, the bifidobacterial population in non-secretor individuals was less tolerant for the harsh conditions of TNO TIM-1 model, mimicking the stomach and small environments.

TABLE 9
The survival of bifidobacteria from pooled faecal samples of
secretor and non-secretor individuals in the TIM-1 model (upper
gastrointestinal tract conditions). The survival was determined
by plate count culturing using Beerens and RB media.

| | Secretor pool | | Non-secretor pool | |
|---|---|---|---|---|
| | Beerens | RB | Beerens | RB |
| Intake, total cfu in | 6.5E+08 | 3.4E+09 | 4.1E+08 | 3.2E+09 |
| sample | | | | |
| Total survival, total | 2.1E+07 | 1.6E+08 | 5.5E+05 | 6.4E+07 |
| cfu in samples | | | | |
| % survival | 3.2 | 4.8 | 0.1. | 2.0 |

1-23. (canceled)

24. A microbial or probiotic composition which is tailored based on the bifidobacterial composition found in the intestine of at least one individual with secretor blood group phenotype but not commonly found in an individual with non-secretor blood group phenotype.

25. The composition according to claim 24, wherein it comprises at least one of the strains listed in Table 4.

26. The composition according to claim 25, wherein it comprises Bifidobacterium bifidum, together with at least one of the strains listed in Table 4.

27. The composition according to claim 24, wherein it comprises at least one of the strains listed in Table 1.

28. The composition according to claim 27, wherein it comprises Bifidobacterium bifidum, together with at least one of the strains listed in Table 1.

29. The composition according to claim 24, wherein said composition additionally includes at least one prebiotic.

30. The composition according to claim 29, wherein the prebiotic includes fucose-containing glycans.

31. The probiotic composition according to claim 29, wherein the prebiotic is lacto-N-biose I.

32. A method of tailoring a microbial or probiotic composition based on the spectrum of bifidobacteria found in the intestine of at least one individual with secretor blood group phenotype but not commonly found in an individual with non-secretor blood group phenotype.

33. A method of assessing the need of an individual for bifidobacteria enriched probiotic supplementation by determining the secretor/non secretor blood group status of the individual.

34. A method of assessing the need of a brest-feeded or weaning baby for bifidobacteria enriched probiotic supplementation by determining the secretor/non secretor blood group status of the baby and that of the baby's mother.

35. A method for estimating a dose of bifidobacteria supplementation needed for a desired effect in an individual by determining the secretor/non secretor blood group status of said individual.

36. A method of identifying an individual at risk for suffering from a gastrointestinal disorder by determining the secretor/non-secretor blood group status of said individual.

37. The composition according to claim 24, wherein the composition is tailored for infants of the non-secretor type.

38. The composition according to claim 24, wherein the composition is tailored for infants regardless of their secretor phenotype, whose breast-feeding mother is of the non-secretor blood group type.

39. The composition according to claim 24, wherein the composition is tailored to elderly individuals for supporting the maintenance of bifidobacteria diversity and abundance.

40. The probiotic composition according to claim 24 for the treatment and/or prevention of inflammatory bowel disease, diarrhoea, respiratory tract infections, irritable bowel syndrome and/or atopy/allergy.
